(19) United States
(12) Patent Application Publication
(10) Pub. No.: US 2022/0366771 A1
(43) Pub. Date: Nov. 17, 2022

(54) Wearable Device for Creating Social Distancing Awareness

(71) Applicant: Linda Morris, Los Angeles, CA (US)

(72) Inventor: Linda Morris, Los Angeles, CA (US)

(21) Appl. No.: 17/302,897

(22) Filed: May 14, 2021

(51) Int. Cl. G08B 21/02; G06F 1/16; G06K 9/00; G16H 10/60

(52) U.S. Cl. CPC G08B 21/02; G06F 1/163; G06K 9/00664; G16H 10/60

(57) ABSTRACT

A wearable device for creating social distancing awareness is disclosed. The wearable device may include a sensor unit, an output unit, and a control unit connected to the sensor unit and the output unit. The sensor unit may detect one or more objects within a predetermined distance from a user wearing the wearable device. The control unit may control the output unit to generate one or more alerts for a predetermined time period, in response to detecting the one or more objects. The output unit may generate the one or more alerts for creating the social distancing awareness.

### TECHNOLOGICAL FIELD

The present disclosure generally relates to wearable devices and more particularly relates to a wearable device for creating social distancing awareness.

### BACKGROUND

The emergence of viral diseases (such as coronavirus related Covid-19) has put lives of public under severe threat, as these viral diseases spread at faster rate and create respiratory problems, which are sometimes fatal. For instance, an uninfected person may be infected with the viral diseases in a short amount of time, when the uninfected person stays in vicinity of an infected person. One of the most known solutions to avoid the spread of the viral diseases is maintaining social distancing between individuals. However, maintaining social distancing does not guarantee that the infected person will act responsibly and there are high chances of the uninfected person catching the infection due to callous or irresponsible behaviour of the infected person. Moreover, it is human tendency to get easily distracted when in presence of more individuals, thus, continuously monitoring one's surroundings to maintain a minimum physical distance from other individuals or objects becomes prone to unreliability and unsafe behaviour.

Accordingly, there is a need for a system to create a robust, reliable, and safe measure providing social distancing awareness to a user, and thus enable preventing the spread of viral diseases.

### BRIEF SUMMARY

In order to solve the foregoing problem, the present disclosure provides a wearable device for providing social distancing awareness to a user wearing the wearable device such that the user need not restlessly monitor his/her surrounding to maintain a minimum physical distance from other users or objects. According to some embodiments, the wearable device may include a sensor unit that is configured to detect one or more objects that are within a predetermined distance from the user. The one or more objects may include one or more of moving objects, a static object, an electronic object, a non-electronic object, and the like. In some example embodiments, the one or more objects may not actively participate in the detection process. In these embodiments, the sensor unit may be configured to detect the one or more objects regardless of electronics associated with the one or more objects.

The wearable device may further include a control unit that is configured to control an output unit to generate one or more alerts, when the one or more objects are detected within the predetermined distance from the user. In an example embodiment, the one or more alerts may include a vibration alert, an LED (light emitting diode) alert, or a combination thereof. The one or more alerts may be generated for a predetermined time period. The one or more alerts may provide an indication, to the user, to move away from the one or more objects. Thereby, the wearable device may aid in creating social distancing awareness by generating the one or more alerts, when the one or more objects are detected within the predetermined distance from the user.

According to some embodiments, the control unit may be further configured to move the wearable device to a reset state, when the one or more objects are not detected. In an example embodiment, when the wearable device is in the reset state, the output unit of the wearable device may terminate the generation of the one or more alerts.

According to some embodiments, the control unit may be further configured to cause the wearable device to move to a hibernated condition, when the one or more objects are not detected for a first predefined time period. The hibernated condition may be indicative of an inactive state of the wearable device. When the wearable device is in the inactive state, the wearable device may consume negligible amount of power (e.g., at most zero watt of power). Thereby, moving the wearable device to the hibernated condition may improve performance of a battery associated with the wearable device.

According to some embodiments, the control unit may be further configured to cause the wearable device to change state, from the hibernated condition to an active state after a second predefined time period. When the wearable device is in the active state, the wearable device is configured to generate the one or more alerts, when the one or more objects are detected within the predetermined distance from the user.

In this way, when the one or more objects are detected within the predetermined distance from the user, the wearable device may aid in creating social distancing awareness by generating the one or more alerts such that the user need not restlessly monitor his/her surrounding to maintain the predetermined distance from the one or more objects.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF DRAWINGS

Having thus described example embodiments of the invention in general terms, reference will now be made to the accompanying drawings, which are not necessarily drawn to scale, and wherein:

FIG. 1 illustrates an exploded view of a wearable device for providing social distancing awareness, in accordance with one or more example embodiments of the present disclosure.

FIG. 2 illustrates a block diagram of the wearable device of FIG. 1, in accordance with the one or more example embodiments of the present disclosure.

FIG. 3 illustrates a configuration of the wearable device of FIG. 1, in accordance with the one or more example embodiments of the present disclosure.

FIG. 4 illustrates a flowchart showing a method for creating social distancing awareness, in accordance with the one or more example embodiments of the present disclosure.

FIG. 5 illustrates an exemplary working scenario showing the wearable device for creating social distancing awareness, in accordance with the one or more example embodiments of the present disclosure.

### DETAILED DESCRIPTION

In the following description, for purposes of explanation, numerous specific details are set forth in order to provide a thorough understanding of the present disclosure. It will be apparent, however, to one skilled in the art that the present disclosure may be practiced without these specific details. In other instances, apparatuses and methods are shown in block diagram form only in order to avoid obscuring the present disclosure.

Reference in this specification to “one embodiment” or “an embodiment” means that a particular feature, structure, or characteristic described in connection with the embodiment is included in at least one embodiment of the present disclosure. The appearance of the phrase “in one embodiment” in various places in the specification are not necessarily all referring to the same embodiment, nor are separate or alternative embodiments mutually exclusive of other embodiments. Further, the terms “a” and “an” herein do not denote a limitation of quantity, but rather denote the presence of at least one of the referenced items. Moreover, various features are described which may be exhibited by some embodiments and not by others. Similarly, various requirements are described which may be requirements for some embodiments but not for other embodiments.

Some embodiments of the present invention will now be described more fully hereinafter with reference to the accompanying drawings, in which some, but not all, embodiments of the invention are shown. Indeed, various embodiments of the invention may be embodied in many different forms and should not be construed as limited to the embodiments set forth herein; rather, these embodiments are provided so that this disclosure will satisfy applicable legal requirements. Like reference numerals refer to like elements throughout. As used herein, the terms “data,” “content,” “information,” and similar terms may be used interchangeably to refer to data capable of being transmitted, received and/or stored in accordance with embodiments of the present invention. Thus, use of any such terms should not be taken to limit the spirit and scope of embodiments of the present invention.

Additionally, as used herein, the term ‘circuitry’ may refer to (a) hardware-only circuit implementations (for example, implementations in analog circuitry and/or digital circuitry); (b) combinations of circuits and computer program product(s) comprising software and/or firmware instructions stored on one or more computer readable memories that work together to cause an apparatus to perform one or more functions described herein; and (c) circuits, such as, for example, a microprocessor(s) or a portion of a microprocessor(s), that require software or firmware for operation even if the software or firmware is not physically present. This definition of ‘circuitry’ applies to all uses of this term herein, including in any claims. As a further example, as used herein, the term ‘circuitry’ also includes an implementation comprising one or more processors and/or portion(s) thereof and accompanying software and/or firmware. As another example, the term ‘circuitry’ as used herein also includes, for example, a baseband integrated circuit or applications processor integrated circuit for a mobile phone or a similar integrated circuit in a server, a cellular network device, other network device, and/or other computing device.

As defined herein, a “computer-readable storage medium,” which refers to a non-transitory physical storage medium (for example, volatile or non-volatile memory device), may be differentiated from a “computer-readable transmission medium,” which refers to an electromagnetic signal.

The embodiments are described herein for illustrative purposes and are subject to many variations. It is understood that various omissions and substitutions of equivalents are contemplated as circumstances may suggest or render expedient but are intended to cover the application or implementation without departing from the spirit or the scope of the present disclosure. Further, it is to be understood that the phraseology and terminology employed herein are for the purpose of the description and should not be regarded as limiting. Any heading utilized within this description is for convenience only and has no legal or limiting effect.

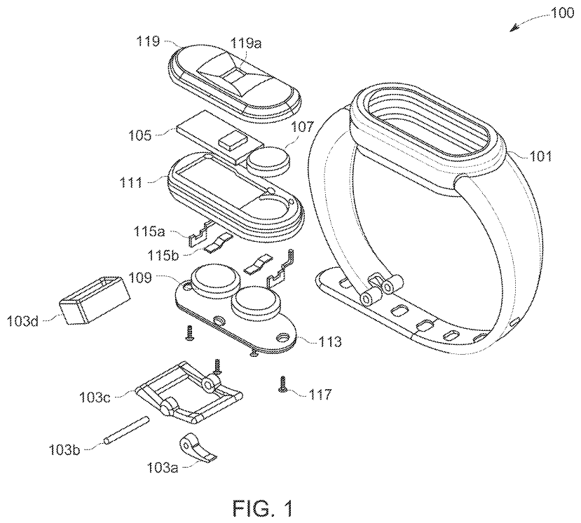

FIG. 1 illustrates an exploded view of a wearable device 100, in accordance with one or more example embodiments of the present disclosure. The wearable device 100 may correspond to a wrist band. The wearable device 100 may include a wrist belt 101 that enables the wearable device 100 to be attached with a user (e.g., a human being). For instance, the wrist belt 101 may be made up of an elastic resin. The wrist belt 101 may be associated with wrist belt elements 103a, 103b, 103c, and 103d. The association of the wrist belt elements 103a, 103b, 103c, and 103d with the wrist belt 101 enables the wearable device 100 to be attached with the user regardless of wrist size of the user.

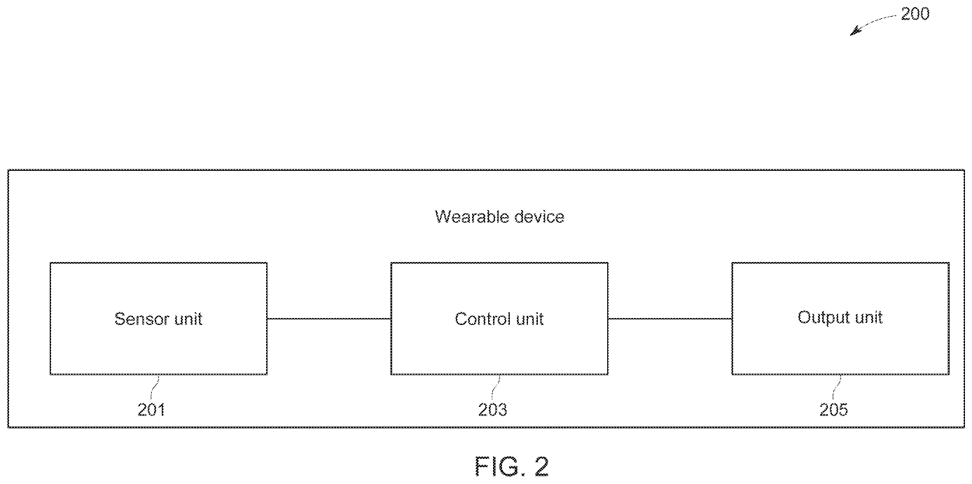

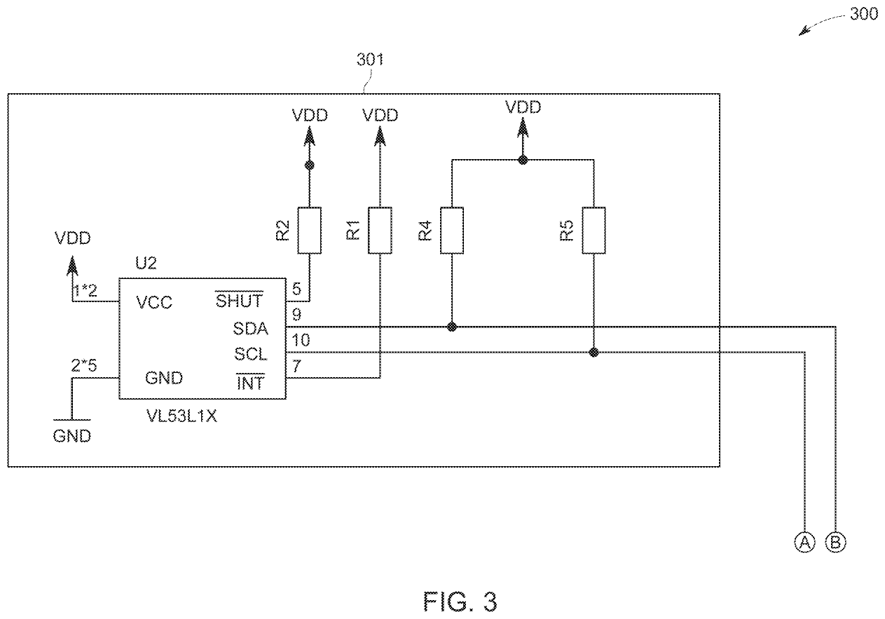

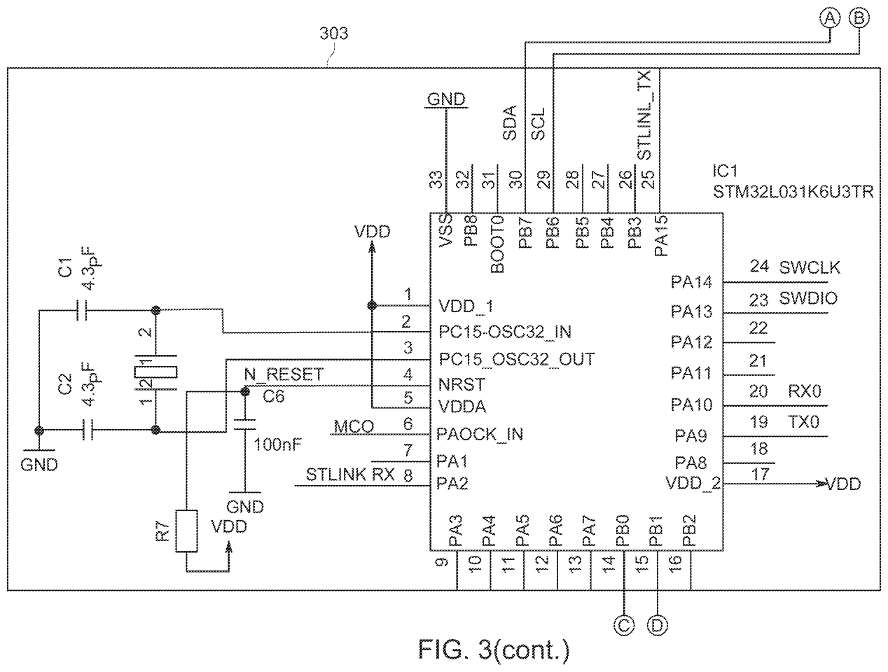

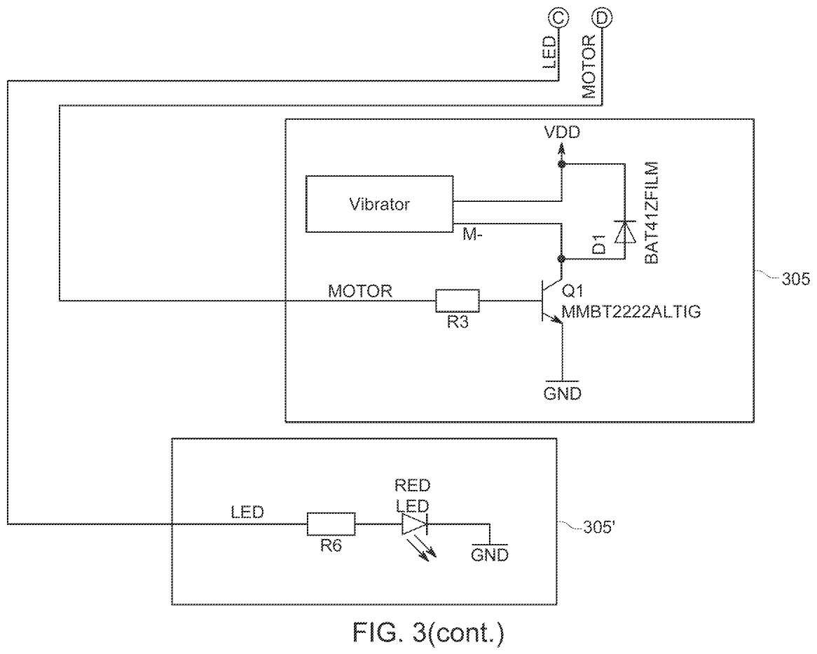

The wearable device 100 may further include electronics part. The electronics part includes at least a Printed Circuit Board (PCB) 105, a vibrator 107, and batteries 109. The electronics part of the wearable device 100 enables the wearable device 100 to create social distancing awareness alerts for the user wearing the wearable device 100. The wearable device 100 may further include a first housing part 111 and a second housing part 113. The first housing part 111 may house the PCB 105 and the vibrator 107 of the electronics part. The second housing part 113 may house the batteries 109 of the electronics part. For instance, the batteries 109 may be coin batteries, which may supply power to the PCB 105 and/or the vibrator 107. The second housing part 113 may include housing elements 115a and 115b to discharge (or charge) the power from the batteries 109. The housing elements 115a and 115b may be made up of a sheet metal. The second housing part 113 may further include metal screws 117 that enable the second housing part 113 to be attached with the first housing part 111. The wearable device 100 may further include a cap 119. The cap 119 may enclose the PCB 105 and the vibrator 107, while leaving an opening 119a. The opening 119a enables to detect one or more objects. The electronics part of the wearable device 100 is further explained in the detailed description of FIG. 2 and FIG. 3.

FIG. 2 illustrates a block diagram of a wearable device 200, in accordance with the one or more example embodiments of the present disclosure. FIG. 2 is explained in conjunction with FIG. 1. The wearable device 200 may correspond to the wearable device 100. The wearable device 200 may include a sensor unit 201, a control unit 203, and an output unit 205. The sensor unit 201 and the control unit 203 may be embodied within the PCB 105. The output unit 205 may correspond to the vibrator 107. The control unit 203 may be connected to the sensor unit 201 and the output unit 205.

According to an embodiment, the sensor unit 201 is configured to detect one or more objects. The one or more objects may include one or more of moving objects, a static object, an electronic object, a non-electronic object, and the like. For instance, the moving object may correspond to a human being under motion, a moving vehicle, or the like. For instance, the static object may correspond to a static human being (e.g., a human being sitting on a chair or the like), a vehicle that is static, a building, or the like. For instance, the electronic object may correspond to any one of: the moving object or the static object that is associated with electronics. For instance, the electronics may include circuits, processors, and the like. For instance, the non-electronic object may correspond to any one of: the moving object or the static object that is not associated with any electronics, sensors, or any other active communication component.

In an example embodiment, the sensor unit 201 is configured to detect the one or more objects that are within a predetermined distance from the user (e.g., the human being) wearing the wearable device 200. For instance, the predetermined distance may be six feet, as prescribed by health based social distancing norms. According to an embodiment, in response to detecting the one or more objects in vicinity of the user (within the predetermined distance) by the sensor unit 201, the control unit 203 is configured to control the output unit 205 to generate one or more alerts for a predetermined time period. Accordingly, the output unit 205 is configured to generate the one or more alerts for the predetermined time period. For instance, the predetermined time period may be a time period of two seconds. For instance, the one or more alerts may include a vibrator alert, a Light Emitting Diode (LED) based alert, or a combination thereof.

The one or more alerts may be an indication, to the user wearing the wearable device 200, indicating that the one or more objects are within the predetermined distance from the user. Thereby, the wearable device 200 may aid in creating social distancing awareness for the user wearing the wearable device 200, by generating the one or more alerts when the one or more objects are within the predetermined distance from the user. For example, when the user wearing the wearable device 200 is standing in a crowd of other individuals, when another individual comes within six feet of the user, the wearable device 200 may start vibrating. This way the user gets to know that their social distancing norm is violated, and they can take appropriate measures to be within a safe distance from the other individual again. All this while, the other individual is not alerted and/or disturbed. So, the user wearing the wearable device 200 may take appropriate actions for their safety in a non-intrusive manner, and the control of their safety lies totally in their own hands, without disturbing other individuals around them.

According to an embodiment, the control unit 203 is further configured to control the wearable device 200 to move to a reset state, when the one or more objects are not detected. In an example embodiment, when the wearable device 200 moves to the reset state, the output unit 205 may be configured to terminate the generation of the one or more alerts. Alternatively, the generation of the one or more alerts may be terminated after the predetermined time period of two seconds.

According to an embodiment, the control unit 203 is further configured to control the wearable device 200 to move to a hibernated condition, when the one or more objects are not detected for a first predefined time period. For instance, the first predefined time period may be a time period of one minute. The hibernated condition is indicative of an inactive state of the wearable device 200. When the wearable device 200 is in the inactive state, power requirements associated with the wearable device 200 may be less. For instance, when the wearable device 200 is in the inactive state, the wearable device 200 may consume almost zero watts of power. Thereby, moving the wearable device 200 to the hibernated condition enables to improve a performance (e.g., battery life) of the batteries 109.

Once the wearable device 200 is moved to hibernated condition, the control unit 203 is further configured to control, after a second predefined time period, the wearable device 200 to change the state associated with the wearable device 200 from the inactive state (i.e., the hibernated condition) to an active state. For instance, when the wearable device 200 is in the active state, the wearable device 200 may generate one or more alerts to the user, in response to detecting the one or more objects within the predetermined distance from the user.

In this way, the wearable device 200 may aid in creating social distancing awareness to the user wearing the wearable device 200, by generating the one or more alerts when the one or more objects are within the predetermined distance from the user. Further, a configuration of the wearable device 200 is as explained in the detailed description of FIG. 3.

FIG. 3 illustrates a configuration of a wearable device 300, in accordance with the one or more example embodiments of the present disclosure. FIG. 3 is explained in conjunction with FIG. 2. The wearable device 300 may correspond to the wearable device 200. As illustrated in FIG. 3, the wearable device 300 may include a sensor circuit 301, a control circuit 303, a first output circuit 305, and a second output circuit 305′. The sensor circuit 301 may correspond to the sensor unit 201. The control circuit 303 may correspond to the control unit 203. The first output circuit 305 and the second output circuit 305′ may correspond to the output unit 205.

For example, the sensor circuit 301 may be embodied using a long-distance ranging Time-of-Flight (ToF) sensor (e.g., a VL53L1X sensor). The sensor circuit 301 may be configured as shown in FIG. 3. The control circuit 303 may be a micro-control-based circuit. For instance, the control circuit 303 may be embodied using an Advanced RISC Machine (ARM) micro-controller (e.g., STM32L031K6U3TR micro-controller). The control circuit 303 may be configured as shown in FIG. 3. For instance, the STM32L031K6U3TR micro-controller is a 33 pin Integrated circuit (IC), where a pin 29 and a pin 30 of the STM32L031K6U3TR micro-controller are connected to a clock line (e.g., SCL (pin 10)) of the VL53L1X sensor and a data line (e.g., SDA (pin 9)) of the VL53L1X sensor, respectively. Further, a pin 4, a pin 6, a pin 8, a pin 19, a pin 20, a pin 23, a pin 24, and a pin 25 of the STM32L031K6U3TR micro-controller are connected to the PCB (e.g., the PCB 105), via a pin header (e.g., a SV2 pin header). Furthermore, a pin 14 and/or a pin 15 of the STM32L031K6U3TR micro-controller are connected to the second output circuit 305′ and/or the first output circuit 305, respectively.

The first output circuit 305 may be a vibrator circuit. For instance, the first output circuit 305 may include a vibrator (e.g., the vibrator 107) and a driver circuit that drives the vibrator. The first output circuit 305 may be configured as shown in FIG. 3. The second output circuit 305′ may be an LED circuit. The second output circuit 305′ may be configured as shown in FIG. 3.

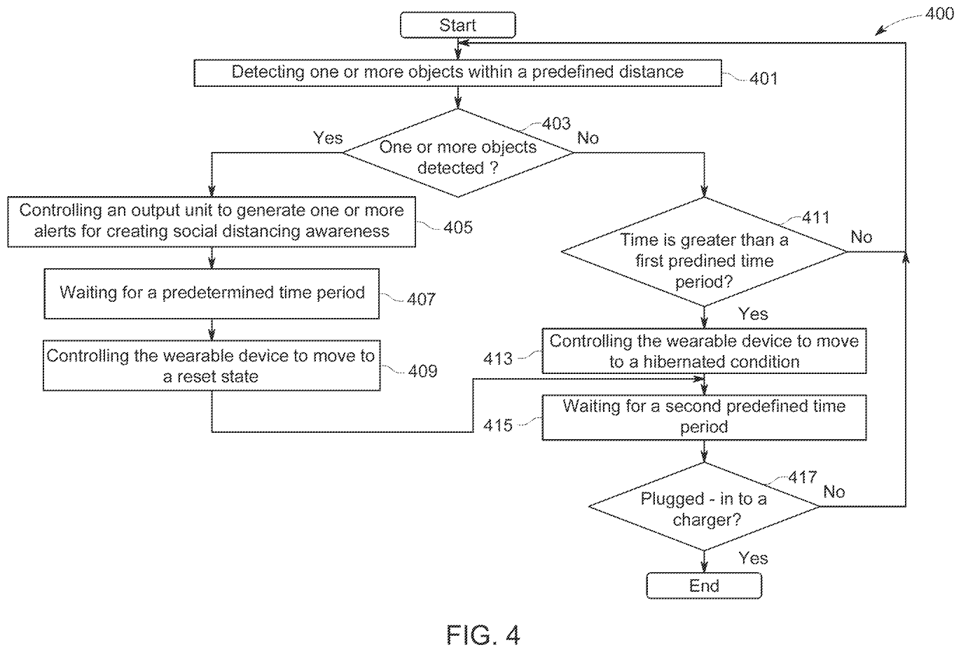

For purpose of explanation, in FIG. 3, the sensor unit 201 is implemented using the sensor circuit 301, the control unit 203 is implemented using the control circuit 303, the output unit 205 is implemented using at least one of the first output circuit 305 and/or the second output circuit 305′ are considered. However, in some implementations, the sensor unit 201 may be implemented using some other circuit like the sensor circuit 301, the control unit 203 may be implemented using some other circuit like the control unit 303. Further, a method implemented by the wearable device 200 is as explained in the detailed description of FIG. 4.

FIG. 4 illustrates a flowchart showing a method 400 for creating social distancing awareness, in accordance with the one or more example embodiments of the present disclosure. The method 400 may be used in conjunction with the wearable device 200 described in the detailed description of FIG. 2. Although various steps of the method 400 are described below and depicted in FIG. 4, the steps need not necessarily all be performed, and in some cases may be performed in a different order than the order shown.

Starting at step 401, the method 400 may include detecting the one or more objects within the predetermined distance from the user wearing the wearable device 200. For instance, the sensor unit 201 may detect the one or more objects that are within the predetermined distance of six feet from the user wearing the wearable device 200. Additionally, at step 401, the method 400 may further include setting a time to zero, before detecting the one or more objects. For instance, the control unit 203 may set a timer to zero.

At step 403, the method 400 may include checking whether the one or more objects are detected. For instance, the control unit 203 may check whether the one or more objects are detected.

If the one or more objects are detected, the method 400 may continue with step 405. At step 405, the method 400 may include controlling the output unit 205 to generate the one or more alerts for creating the social distancing awareness. For instance, the control unit 203 may control the output unit 205 to generate the one or more alerts, like a vibration alert or an LED light blinking alert, for creating the social distancing awareness.

At step 407, the method 400 may include waiting for the predetermined time period. For instance, the control unit 203 may be provided with a delay of the predetermined time period.

At step 409, the method 400 may include controlling the wearable device 200 to move to the reset state. For instance, the control unit 203 may control the wearable device 200 to move to the reset state. For instance, the control unit 203 may reset the sensor unit 201, the output unit 205, and/or the timer. Further, the method 400 may proceed with step 415. At step 415, the method 400 may include waiting for a second predefined time period before again returning to step 401 to be able to again detect the one or more objects. For instance, the sensor unit 201 and/or the control unit 203 may remain in the inactive state for the second predefined time period. Next, the method may proceed to step 417.

At step 417, the method 400 may include checking whether the wearable device 200 is plugged-in to a charger. For instance, the control unit 203 may check whether the wearable device 200 is plugged-in to the charger. If the wearable device 200 is not plugged-in to the charger, the method 400 may return control to step 401. At step 401, the method may then again start with another iteration of detecting the one or more objects.

At step 403, if the one or more objects are not detected, the method 400 may proceed with step 411. At step 411, the method 400 may include checking whether the time, such as time elapsed, is equal to or greater than the first predefined time period. For instance, the control unit 203 may check whether the time is equal to or greater than the first predefined time period of one minute. If the time is less than the first predefined time period, the method 400 may proceed with step 401. If the time is equal to or greater than the first predefined time period, the method 400 may proceed with step 413.

At step 413, the method 400 may include controlling the wearable device 200 to move to the hibernated condition. For instance, the control unit 203 may control the wearable device 200 to move to the hibernated condition.

At step 415, the method 400 may include waiting for a second predefined time period. For instance, the sensor unit 201 and/or the control unit 203 may remain in the inactive state for the second predefined time period.

At step 417, the method 400 may include checking whether the wearable device 200 is plugged-in to a charger. For instance, the control unit 203 may check whether the wearable device 200 is plugged-in to the charger. If the wearable device 200 is not plugged-in to the charger, the method 400 may proceed with step 401. If the wearable device 200 is plugged-in to the charger, the wearable device 200 may stop executing the method 400.

On implementing the method 400, the wearable device 200 may aid in creating social distancing awareness for the user wearing the wearable device 200, by generating the one or more alerts when the one or more objects are within the predetermined distance from the user.

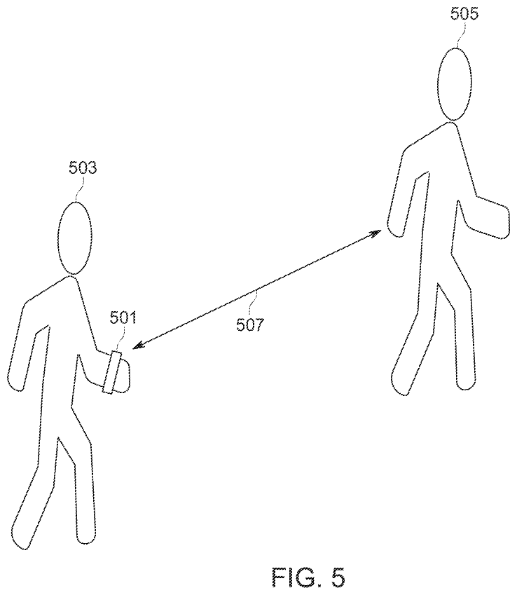

FIG. 5 illustrates an exemplary working scenario showing in which the method 400 may be implemented by a user 503 wearing a wearable device 501 for creating social distancing awareness, in accordance with the one or more example embodiments of the present disclosure. The wearable device 501 may correspond to the wearable device 200. As illustrated in FIG. 5, the wearable device 501 may be the wrist band associated with the user 503. However, as may be understood by a person of ordinary skill in the art that the wearable device 501 may also be implemented in the form of any other wearable configuration, such as a ring, a watch, a headband, a pair of glasses, and the like, without deviating the scope of the present invention. According to an embodiment, the wearable device 501 may be configured to generate, for the user 503, the one or more alerts for creating social distancing awareness, when the wearable device 501 detects a user 505 different from the user 503 within a predetermined distance 507 (e.g., six feet) from the user 503. The user 505 may correspond to the one or more objects. According to some embodiments, the wearable device 501 may detect the user 505 regardless of electronics associated with the user 505. Specifically, the sensor unit (e.g., the sensor unit 201) detects the user 505 regardless of the electronics associated with the user 505. That is to say, the user 505 does not need to have any active electronic elements in their possession for detection. To this end, when the user 505 is within the predetermined distance 507 from the user 503, the wearable device 501 detects the user 505 even if the user 505 is not wearing another wearable device (that is identical to the wearable device 501). In other words, when the user 505 is within the predetermined distance 507 from the user 503, the wearable device 501 detects the user 505 even if the user 505 is not associated with the wearable device 501 and/or not associated with another wearable device. Accordingly, the user 505 passively participates in the detection, when the user 505 is at a distance less than or equal to the predetermined distance 507 from the user 503. In other words, the user 505 is not actively participating in the detection, as the wearable device 501 detects the user 505 regardless of electronics associated with the user 505.

Further, the wearable device 501 generates the one or more alerts for the user 503 wearing the wearable device 501 without annoying the user 505, when the user 505 is detected within the predetermined distance 507 from the user 503. The one or more alerts may be the indication, to the user 503, to move away from the user 505 such that social distancing between the user 503 and the user 505 is maintained. Thereby, the wearable device 501 may break a chain of spreading viral diseases such as coronavirus based Covid-19 disease.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Moreover, although the foregoing descriptions and the associated drawings describe example embodiments in the context of certain example combinations of elements and/or functions, it should be appreciated that different combinations of elements and/or functions may be provided by alternative embodiments without departing from the scope of the appended claims. In this regard, for example, different combinations of elements and/or functions than those explicitly described above are also contemplated as may be set forth in some of the appended claims. Although specific terms are employed herein, they are used in a generic and descriptive sense only and not for purposes of limitation.

We claim:

1. A wearable device for creating social distancing awareness, the wearable device comprising:
a sensor unit configured to detect one or more objects within a predetermined distance from a user wearing the wearable device;
an output unit configured to generate one or more alerts for creating the social distancing awareness; and
a control unit connected to the sensor unit and the output unit, wherein the control unit is configured to control the output unit to generate the one or more alerts for a predetermined time period, in response to detecting the one or more objects.

2. The wearable device of claim 1, wherein the one or more objects are not associated with the wearable device or another wearable device.

3. The wearable device of claim 1, wherein the one or more objects include one or more of a moving object, a static object, an electronic object, and a non-electronic object that are not actively participating in the detection.

4. The wearable device of claim 1, wherein the sensor unit is further configured to detect the one or more objects regardless of electronics associated with the one or more objects.

5. The wearable device of claim 1, wherein the control unit is further configured to control the wearable device to move to a hibernated condition, when the one or more objects are not detected for a first predefined time period, and wherein the hibernated condition is indicative of an inactive state of the wearable device.

6. The wearable device of claim 5, wherein the control unit is further configured to control the wearable device to change state, from the hibernated condition to an active state after a second predefined time period.

7. The wearable device of claim 1, wherein the control unit is further configured to control the wearable device to move to a reset state when the one or more objects are not detected.

8. The wearable device of claim 7, wherein the output unit is further configured to terminate the generation of the one or more alerts, in response to moving the wearable device to the reset state.

9. The wearable device of claim 1, wherein the one or more alerts comprise a vibration alert, an LED (light emitting diode) alert, or a combination thereof.

10. A method for creating social distancing awareness, the method comprising:
detecting, by a sensor unit, one or more objects within a predetermined distance from a user; and
controlling an output unit, by a control unit connected to the sensor unit and the output unit, to generate one or more alerts for creating social distancing awareness, in response to detecting the one or more objects, wherein the one or more alerts are generated for a predetermined time period.

11. The method of claim 10, wherein the one or more objects are not associated with the wearable device or another wearable device.

12. The method of claim 10, wherein the one or more objects include one or more of a moving object, a static object, an electronic object, and a non-electronic object that are not actively participating in the detection.

13. The method of claim 10, further comprising detecting, by the sensor unit, the one or more objects regardless of electronics associated with the one or more objects.

14. The method of claim 10, further comprising controlling, by the control unit, the wearable device to move to a hibernated condition, when the one or more objects are not detected for a first predefined time period, and wherein the hibernated condition is indicative of an inactive state.

15. The method of claim 14, further comprising controlling, by the control unit, the wearable device to change state from the hibernated condition to an active state after a second predefined time period.

16. The method of claim 10, further comprising controlling, by the control unit, the wearable device to move to a reset state when the one or more objects are not detected.

17. The method of claim 16, further comprising terminating, by the output unit, the generation of the one or more alerts in response to moving the wearable device to the reset state.

18. A computer program product comprising a non-transitory computer readable medium having stored thereon computer executable instruction which when executed by a wearable device, cause the wearable device to carry out operations for creating social distancing awareness, the operations comprising:
detecting, by a sensor unit, one or more objects within a predetermined distance from a user wearing the wearable device; and
controlling, by a control unit connected to the sensor unit and an output unit, the output unit to generate one or more alerts for creating social distancing awareness, in response to detecting the one or more objects, wherein the one or more alerts are generated for a predetermined time period.

19. The computer program product of claim 18, wherein the one or more objects are not associated with the wearable device or another wearable device.

20. The computer program product of claim 18, wherein the one or more objects include one or more of a moving object, a static object, an electronic object, and a non-electronic object that are not actively participating in the detection.
